SEQUENCE LISTING (56 sequences)

SEQ ID NO: 1 - DNA, 20 bp, Artificial
  primer sequence
gggtcttgggtgtaaatgga

SEQ ID NO: 2 - DNA, 20 bp, Artificial
  primer sequence
cacacttggagaccagacga

SEQ ID NO: 3 - DNA, 51 bp, Artificial
  human leukocyte
aacgggatgaagataagaaccagaacgggatttagaacctgtaacaccatg

SEQ ID NO: 4 - DNA, 950 bp, Artificial
  human leukocyte
taattcagtccccagtcctactaggttccccatttgttttgtagggacaacaggagatttaaagggattagtagtcatcaaaggccttgagtttctttgaagagacaacattcaagaacaaagaagaaagttggtttaggagctaaatttcacatatttttggtttggcatgaaacctagacctgcccctccaccaccattacacagttaagtccacagtaatggctttgactctcattaattatatctaattcacatttttcagggtcaataggcagttagtctaacatggaaaaatagaagcagatagatgataaactaaaaccaatcctcactaagaaaaatactgacattgaatctttttcaaactgggcaaagactgagtcaatttagagtaatccatacagacatagtgaacacttacgagaagttaattccacacttggagaccagacgataaatgacaaagtcaaacaacgggatgaagataagaaccagaagggatttagaacctgtaacaccatgataaaaaggattagctacaaatattctcctatcatgagtgtgagcactgcactcatttgcttgtttacactctgtctcacatctacacacacttttgtcattgtaagtacagaatgactccacattcctcagtccatttacacccaagaccctatttcccttgcatagcattttagctttcaaatatcccaagggacaagtttcactctgagcctgtctttctgccagccttattgtgagctctttcctctcatgacttcattgctcacaaaataagccttacaatttcttttacaagattttgattatactatcttgacttggaaaaaaatccaatgcttacacttaacccgttgcaagttaagtacttaacaggggatgtgaaggagaagggtggggcttaatgggaatgttttcatgtcgaa

SEQ ID NO: 5 - DNA, 23 bp, Artificial
  FMO3 primer-F
gatgttaccactgaaagggatgg

SEQ ID NO: 6 - DNA, 23 bp, Artificial
  FMO3 primer-R
gaagcgaccttgtgaatagatgc

SEQ ID NO: 7 - DNA, 23 bp, Artificial
  CYP8B1 primer-F
aagaatgactgtatgcccttcca

SEQ ID NO: 8 - DNA, 23 bp, Artificial
  CYP8B1 primer-R
aagtgtataggcaagcagttggg

SEQ ID NO: 9 - DNA, 23 bp, Artificial
  SLC15A2 primer-F
agggaaatagggtcttgggtgta

SEQ ID NO: 10 - DNA, 23 bp, Artificial
  SLC15A2 primer-R
tctttttcaaactgggcaaagac

SEQ ID NO: 11 - DNA, 23 bp, Artificial
  SLC15A2 primer-F
gctgagtcaaaaagcatcgagtt

SEQ ID NO: 12 - DNA, 23 bp, Artificial
  SLC15A2 primer-R
attgttttcatttcccaccactg

SEQ ID NO: 13 - DNA, 23 bp, Artificial
  SLC15A2 primer-F
ttaccaaggatctgcctgatgat

SEQ ID NO: 14 - DNA, 23 bp, Artificial
  SLC15A2 primer-R
atcttcgaatcccacatgagaaa

SEQ ID NO: 15 - DNA, 23 bp, Artificial
  UGT2B15 primer-F
atggcgacacgtcttcaaaatag

SEQ ID NO: 16 - DNA, 23 bp, Artificial
  UGT2B15 primer-R
gggagaaagggagaaaaacaaaa

SEQ ID NO: 17 - DNA, 23 bp, Artificial
  DDR1 primer-F
agatggactcctgtcttacaccg

SEQ ID NO: 18 - DNA, 23 bp, Artificial
  DDR1 primer-R
gggtgcctttttcatacagtgtc

SEQ ID NO: 19 - DNA, 23 bp, Artificial
  DDR1 primer-F
ctagagagaacaatggcagagcc

SEQ ID NO: 20 - DNA, 22 bp, Artificial
  DDR1 primer-R
cactgaggaactggttgaggtc

SEQ ID NO: 21 - DNA, 23 bp, Artificial
  ABCB1 primer-F
acaatggcctgaaaactgaaaaa

SEQ ID NO: 22 - DNA, 23 bp, Artificial
  ABCB1 primer-R
cattgcaatagcaggagttgttg

SEQ ID NO: 23 - DNA, 23 bp, Artificial
  PON3 primer-F
tcctacctcaattcctcagatgg

SEQ ID NO: 24 - DNA, 23 bp, Artificial
  PON3 primer-R
ccgtttcctgtcttttccttctt

SEQ ID NO: 25 - DNA, 23 bp, Artificial
  PDGFRL primer-F
aagcaaaacgaagatgtcagagg

SEQ ID NO: 26 - DNA, 23 bp, Artificial
  PDGFRL primer-R
caaatcaggatgaactcccaaag

SEQ ID NO: 27 - DNA, 23 bp, Artificial
  PDGFRL primer-F
aaacctgggagtcctcaacctta

SEQ ID NO: 28 - DNA, 23 bp, Artificial
  PDGFRL primer-R
aggaactgaggtccagagaggac

SEQ ID NO: 29 - DNA, 23 bp, Artificial
  PDGFRL primer-F
cgtgcattggcacaatatatcac

SEQ ID NO: 30 - DNA, 23 bp, Artificial
  PDGFRL primer-R
gaccacacactgtcttctgttgc

SEQ ID NO: 31 - DNA, 23 bp, Artificial
  PDGFRL primer-F
tgacactcacctacaaaagcagg

SEQ ID NO: 32 - DNA, 23 bp, Artificial
  PDGFRL primer-R
tccttgctaaaacaccactgtga

SEQ ID NO: 33 - DNA, 23 bp, Artificial
  PDGFRL primer-F
atgtcctccttccctgatctacc

SEQ ID NO: 34 - DNA, 23 bp, Artificial
  PDGFRL primer-R
ttatcagagaggaagatggctgc

SEQ ID NO: 35 - DNA, 23 bp, Artificial
  PDGFRL primer-F
ctttgggagttcatcctgatttg

SEQ ID NO: 36 - DNA, 23 bp, Artificial
  PDGFRL primer-R
gtgatatattgtgccaatgcacg

SEQ ID NO: 37 - DNA, 23 bp, Artificial
  PDGFRL primer-F
tgacactcacctacaaaagcagg

SEQ ID NO: 38 - DNA, 23 bp, Artificial
  PDGFRL primer-R
tccttgctaaaacaccactgtga

SEQ ID NO: 39 - DNA, 23 bp, Artificial
  PDGFRL primer-F
tccaagttccacttgagttttcc

SEQ ID NO: 40 - DNA, 23 bp, Artificial
  PDGFRL primer-R
gctcttgtttgtttaggtccagg

SEQ ID NO: 41 - DNA, 23 bp, Artificial
  PDGFRL primer-F
cgtgcattggcacaatatatcac

SEQ ID NO: 42 - DNA, 23 bp, Artificial
  PDGFRL primer-R
tgtcttctgttgctctgtccttg

SEQ ID NO: 43 - DNA, 23 bp, Artificial
  MUSK primer-F
acacagaatttaggctctgccac

SEQ ID NO: 44 - DNA, 23 bp, Artificial
  MUSK primer-R
ccaaagtcttgggagaactctgt

SEQ ID NO: 45 - DNA, 23 bp, Artificial
  ALDH3B1 primer-F
tgaggctcagaggggagaagtag

SEQ ID NO: 46 - DNA, 23 bp, Artificial
  ALDH3B1 primer-R
acagctgtcatggtggtctacag

SEQ ID NO: 47 - DNA, 23 bp, Artificial
  ALDH3B1 primer-F
tgaggctcagaggggagaagtag

SEQ ID NO: 48 - DNA, 23 bp, Artificial
  ALDH3B1 primer-R
acagctgtcatggtggtctacag

SEQ ID NO: 49 - DNA, 23 bp, Artificial
  FLT1 primer-F
acatgctgtgtcagcaccttcta

SEQ ID NO: 50 - DNA, 23 bp, Artificial
  FLT1 primer-R
accagtttctagaccaggggtgt

SEQ ID NO: 51 - DNA, 23 bp, Artificial
  ALDH6A1 primer-F
gtgattggttaggagcgaaaatg

SEQ ID NO: 52 - DNA, 23 bp, Artificial
  ALDH6A1 primer-R
caaagagaaaccctatccccaac

SEQ ID NO: 53 - DNA, 23 bp, Artificial
  ALDH6A1 primer-F
gtgattggttaggagcgaaaatg

SEQ ID NO: 54 - DNA, 23 bp, Artificial
  ALDH6A1 primer-R
caaagagaaaccctatccccaac

SEQ ID NO: 55 - DNA, 23 bp, Artificial
  ALDH6A1 primer-F
ctttcttgggctcttctcctttc

SEQ ID NO: 56 - DNA, 23 bp, Artificial
  ALDH6A1 primer-R
ggtttgtgagaatcattccatcc

(19) United States
(12) Patent Application Publication
(10) Pub. No.: US 2017/0211147 A1
(43) Pub. Date: Jul. 27, 2017

(54) METHOD OF PREDICTING REACTION TO SORAFENIB TREATMENT USING GENE POLYMORPHISM

(71) Applicant: National Cancer Center, Gyeonggi-do (KR)

(72) Inventors: Joong-Won PAKR, Gyeonggi-do (KR); Yeon Su LEE, Gyeonggi-do (KR); Bo Hyun KIM, Seoul (KR)

(21) Appl. No.: 15/302,872

(22) PCT Filed: Mar. 23, 2015
(86) PCT No.: PCT/KR2015/002799

§ 371 Date: Oct. 7, 2016

(30) Foreign Application Priority Data
Apr. 9, 2014 (KR) 10-2014-0042571

(51) Int. Cl. C12Q 1/68

(52) U.S. Cl. CPC C12Q 1/6886; C12Q 2600/106; C12Q 2600/156; C12Q 2600/158

(57) ABSTRACT

The present invention relates to a method of predicting reaction to Sorafenib treatment using genetic polymorphism. More specifically, for the reaction to Sorafenib treatment according to the present invention, it is possible to predict the reaction of a test object to Sorafenib treatment by using an anticancer-target gene which is expressed in a biological sample of a liver cancer patient as a biomarker, whereby a proper drug is administered to a liver cancer patient and an optimal treatment effect is attained, so that inconvenience of a patient can be reduced, costs for treatment can be reduced, and an individually tailored chemotherapy can more effectively be implemented by administration of a patient-specific anticancer agent.

### TECHNICAL FIELD

The present invention relates to a method of predicting the response to sorafenib treatment using genetic polymorphism, and more particularly, to a method of predicting the response to sorafenib treatment using genetic polymorphism, which may allow more effective implementation of individually tailored chemotherapy by predicting the response to sorafenib treatment using an anticancer agent target gene expressed in a biological sample obtained from a liver cancer patient as a biomarker.

### BACKGROUND ART

Cancer is one of the most deadly threats to human health, and even in the United States, about 1.3 million new cancer patients are generated annually. Cancer is the second leading cause of death behind cardiovascular diseases, and approximately one of the four deaths is estimated to be a cancer patient. In most cases, such deaths are caused by solid cancer. Although considerable progress has been made in medical treatment for specific cancer, 5-year overall survival rates of all types of cancer have only increased approximately 10% over the past two decades. Since cancer or a malignant tumor is rapidly developed and grown in an uncontrolled manner, it is ultimately difficult to detect and treat it at a proper time.

Today, for cancer treatment, surgery, radiation therapy, chemotherapy, etc. are used.

Currently, approximately 60 types of various anticancer agents are used, and recently, as knowledge on the cancer occurrence and the characteristics of cancer cells becomes very well known, research on the development of a new anticancer agent is actively being conducted. However, when an anticancer agent is repeatedly administered for a long period of time, or cancer reoccurs, cancer cells acquire a tolerance to the anticancer agent, thereby losing a therapeutic effect. Also, most of the anticancer agents exhibit effects by inhibiting the synthesis of a nucleic acid in cells or directly binding to a nucleic acid to damage their function, but these anticancer agents do not selectively act on cancer cells and damage normal cells, particularly, tissue cells in which cell division is actively performed, and thus have a variety of side effects such as bone marrow function degradation, damage to the mucous membrane of the gastrointestinal tract, hair loss, etc.

Therefore, due to the tolerance to such an anticancer agent, there have been constant demands on the development of various types of drugs in the market, and particularly, selective treatment with an anticancer agent is needed to minimize side effects of consumers (patients).

According to conventional cancer chemotherapy, a proper anticancer agent is selected and administered depending on the type and severity of cancer, and not depending on an individual cancer patient. However, overall clinical results have significant differences in the therapeutic effects of such anticancer chemotherapy depending on a patient, and to overcome such differences, various methods are suggested.

To overcome shortcomings of the above-described chemotherapy, there are many attempts to selectively administer a proper anticancer agent by analyzing single-nucleotide polymorphisms (SNPs) per individual. Also, according to the trend of the development of a new anticancer agent, the importance of the development of a target anticancer agent, a biopharmaceutical, a preventive vaccine and a diagnostic agent is increasing, and the development of an oral anticancer agent is increased to enhance the compliance of a cancer patient.

A target anticancer agent does not kill cancer cells. Instead, the target anticancer agent is a drug which inhibits the proliferation and growth of cancer cells by suppressing factors required to grow the cancer cells. For this reason, even in a patient for whom it is difficult to eradicate cancer, cancer progression may be slowed and a survival period may be extended by using the target anticancer agent. Theoretically, since the target anticancer agent does not have toxicity acting on a normal cell, it has less painful side effects. Therefore, in the aspect of the quality of life, an excellent effect is expected, compared to a conventional anticancer agent.

As the prior art on the target anticancer agent, Korean Patent Application Publication No. 10-2013-0058631 (Publication Date: Jun. 4, 2013) discloses a pharmaceutical composition or an anticancer supplement for inhibiting a tolerance to a target anticancer agent, which includes at least one selected from the group consisting of an integrin (33 neutralizing antibody, integrin (33 siRNA, an Src inhibitor and Src siRNA as an active ingredient.

Meanwhile, hepatocellular carcinoma (HCC) is one of the most common types of cancer, particularly, with the high prevalence in Asia, and the third leading cause of death by cancer. For such a type of liver cancer, sorafenib is known as substantially the sole first-line treatment agent for liver cancer.

Sorafenib is known as an oral multikinase inhibitor that simultaneously inhibits receptor tyrosine kinases, which are expected to be overexpressed in tumor cells or tumor vessels, for example, VEGFR-2, platelet-derived growth factor receptor (PDGFR)-β and c-kit, and serine/threonine kinases in a signaling pathway, for example, Raf kinase, and attacks only cancer cells, rather than normal cells, and vascular endothelial cells providing nutrients to the cancer cells so as to treat cancer.

Clinical trials for sorafenib efficacy on various solid tumors are in progress, and sorafenib is already used as a target anticancer agent for renal cell carcinoma. According to the progress of clinical trials on advanced hepatocellular carcinoma, recently, sorafenib was approved by the US Food and Drug Administration (US FDA) as a therapeutic agent for hepatocellular carcinoma, which cannot be removed by excision. Also, sorafenib (Nexavar) generated sales of 373 million euros for the first half of year 2013, has received current approval as therapeutic agents for liver cancer and kidney cancer, and also has been approved lately by the US FDA as a therapeutic agent for thyroid carcinoma.

However, as many patients were identified as unresponsive to sorafenib administration and treatment, there were no methods of predicting and confirming a therapeutic reaction before the initiation of treatment, and due to insufficient research on a reliable biomarker, it was very difficult to predict the response of a subject with respect to sorafenib treatment for administration of a proper drug.

Further, there was insufficient research on a method of predicting the response of a subject with respect to sorafenib treatment to substantially reduce the inconvenience of a patient and reduce treatment costs by administering a proper drug (sorafenib) to a great number of liver cancer patients, thereby achieving an optimal therapeutic effect.

Therefore, the inventors had first validated the usefulness of an SLC15A2 genetic polymorphism as a biomarker indicating the response to sorafenib treatment, and thus completed the present invention.

### DISCLOSURE

### Technical Problem

The present invention has been devised to solve the above-described problems, and the first object to be solved in the present invention is to provide a method of predicting the response to sorafenib treatment which allows more effective implementation of individually tailored chemotherapy by predicting the response.

The second object to be solved in the present invention is to provide a diagnosis kit for predicting the response of a subject with respect to sorafenib treatment, which has excellent effects of reducing side effects of anticancer treatment and treatment costs by predicting the response.

### Technical Solution

To accomplish the first object of the present invention, a method of predicting the response to sorafenib treatment is provided, the method including: obtaining a sample from a subject and detecting the absence or presence of an SLC15A2 genetic polymorphism affecting the response to sorafenib treatment.

According to an exemplary embodiment of the present invention, the SLC15A2 genetic polymorphism may be a C-to-T variation at the 501^{st} nucleotide in the SLC15A2 gene (NCBI ACESSION NO: NM_021082; SEQ. ID. NO: 4).

According to another exemplary embodiment of the present invention, the subject may be a liver cancer patient, and the sample may be blood.

According to still another exemplary embodiment of the present invention, the method of predicting the response to sorafenib treatment includes: obtaining a sample from a subject and determining if the 501^{st} nucleotide in the SLC15A2 gene (NCBI ACESSION NO: NM_021082; SEQ. ID. NO: 4) of the subject has a C/T or T/T genotype; and predicting the response of the subject with respect to the sorafenib treatment based on the determination, and it may be evaluated that the presence of the C/T or T/T genotype shows that a subject has an excellent response to the sorafenib treatment, compared to a subject having a C/C genotype.

According to yet another exemplary embodiment of the present invention, the determining of the genotype may include amplifying the SLC15A2 gene using a set of primers set forth in SEQ. ID. NO: 1 and SEQ. ID. NO: 2, and detecting a nucleotide polymorphism at the 501^{st} nucleotide in the SLC15A2 gene through sequencing.

To accomplish the second object of the present invention, a marker composition for predicting the response to sorafenib treatment is provided, the composition including: an agent for detecting the absence or presence of the SLC15A2 genetic polymorphism affecting the response to sorafenib treatment.

According to an exemplary embodiment of the present invention, the SLC15A2 genetic polymorphism may be a C-to-T variation at the 501^{st} nucleotide in the SLC15A2 gene (NCBI ACESSION NO: NM_021082; SEQ. ID. NO: 4).

According to another exemplary embodiment of the present invention, the agent for detecting the absence or presence of the SLC15A2 genetic polymorphism may include a set of primers set forth in SEQ. ID. NO: 1 and SEQ. ID. NO: 2.

According to still another exemplary embodiment of the present invention, when the 501^{st} nucleotide in the SLC15A2 gene (NCBI ACESSION NO: NM_021082; SEQ. ID. NO: 4) has a C/T or T/T genotype, it may be evaluated that the response to sorafenib is better than a gene having a C/C genotype.

The present invention also provides a diagnosis kit for predicting the response to sorafenib treatment, which includes a marker composition for predicting the response to sorafenib treatment.

According to an exemplary embodiment of the present invention, the diagnosis kit may be an RT-PCR kit or a DNA chip kit.

According to another exemplary embodiment of the present invention, the DNA chip kit may have primers or probes that are immobilized to a substrate, so as to detect a polymorphism at the 501^{st} nucleotide in the SLC15A2 gene (NCBI ACESSION NO: NM_021082; SEQ. ID. NO: 4), and may include a labeling means for detecting hybridization between the DNA chip and a sample.

According to still another exemplary embodiment of the present invention, probes including a positive control hybridized with all nucleotide sequences in the sample and a negative control not hybridized with any nucleotide sequence may be bound to a surface of the substrate.

### Advantageous Effects

The present invention relates to a method of predicting the response to sorafenib treatment so as to minimize side effects of cancer treatment using genetic polymorphism. The response of a liver cancer patient with respect to sorafenib treatment of the present invention can be predicted by using the SLC15A2 gene as a biomarker, and thus a proper drug is administered to the liver cancer patient so as to achieve an optimal therapeutic effect, reduce the inconvenience of the patient, and reduce treatment costs, resulting in an excellent anticancer therapeutic effect and prognosis.

### DESCRIPTION OF DRAWINGS

FIG. 1 shows primer sequences for PCR carried out in Example 1.

FIG. 2 shows diagrams of six non-synonymous SNVs located in four genes, for example, MUSK, ABCB1, FMO3 and SLC15A2 (the arrow represents a variation position; and the number represents an amino acid position).

FIG. 3 shows the progression-free survival time with respect to sorafenib treatment according to SLC15A2 genotypes in liver cancer patients.

FIG. 4 shows the results of Sanger sequencing, in which Hep3B, SNU182 and PLC/PRF5 cell lines having three genotypes are selected, for functional analysis of SLC15A2 genetic polymorphisms in liver cancer cell lines.

FIG. 5 is a graph showing cell viability according to sorafenib treatment through the MTT assay performed on liver cancer cell lines Hep3B, SNU182 and PLC/PRF5.

FIG. 6 shows protein expression according to sorafenib treatment by western blotting performed on liver cancer cell lines Hep3B, SNU182 and PLC/PRF5 (Lane 1: the expression of SLC15A2 gene in PLC/PRF5 cell line, Lane 2: the expression of SLC15A2 gene in Hep3B cell line, and Lane 3: the expression of SLC15A2 gene in SNU182 cell line).

FIG. 7 shows the single-nucleotide polymorphism (SEQ. ID. NO: 3) present at nucleotide 26 in the SLC15A2 gene (NCBI ACESSION NO: NM_021082, SEQ. ID. NO: 4), shown in yellow.

FIG. 8 shows the single-nucleotide polymorphism present at the 501^{st} nucleotide in the SLC15A2 gene (NCBI ACESSION NO: NM_021082, SEQ. ID. NO: 4), shown in fluorescent green.

### MODES OF THE INVENTION

Hereinafter, the present invention will be described in further detail.

As described above, since many liver cancer patients were identified as unresponsive to sorafenib administration and treatment, there were no methods of predicting and confirming the treatment response before initiation of the treatment, and due to insufficient research on a reliable biomarker, it was very difficult to predict the response of a subject with respect to sorafenib treatment in order to administer a proper drug.

Further, there was insufficient research on a method of predicting the response of a subject with respect to sorafenib treatment, which can achieve an optimal therapeutic effect by administering a proper drug (sorafenib) to a great number of liver cancer patients, thereby substantially reducing the inconvenience of a patient and treatment costs.

Therefore, according to an exemplary embodiment of the present invention, a method of predicting the response to sorafenib treatment by obtaining a sample from a subject and detecting the absence or presence of a polymorphism in the SLC15A2 gene affecting the response to the sorafenib treatment was provided to attempt to solve the above-described problem.

Unlike the conventional sorafenib treatment for liver cancer patients in which many patients were identified as unresponsive with respect to sorafenib administration and treatment and thus the treatment responses were not predicted and confirmed before initiation of the treatment, the method according to the present invention may predict the response of a subject with respect to sorafenib treatment, and thus a suitable drug may be administered. Also, as an anticancer-target gene expressed in a biological sample obtained from the liver cancer patient is used as a biomarker, the response to sorafenib treatment for a liver cancer patient may be predicted. Accordingly, a proper drug is administered to the liver cancer patient, thereby achieving an optimal therapeutic effect, the inconvenience of the patient may be reduced, treatment costs may be reduced, and individually tailored chemotherapy may be more effectively implemented by the administration of a patient-specific anticancer agent.

Therefore, the problems of side effects of anticancer treatment caused by conventional cancer chemotherapy in which a proper anticancer agent is selected and administered according to the type and severity of cancer, not according to an individual cancer patient, may be solved.

In the present invention, a variety of single-nucleotide variations (SNVs) and genes associated with sorafenib responses, which can be used as a biomarker for predicting a drug response to sorafenib in a liver cancer patient, were identified, and it was confirmed that, among them, the SLC15A2 genotype plays an important role in the response to the sorafenib treatment in the liver cancer patient.

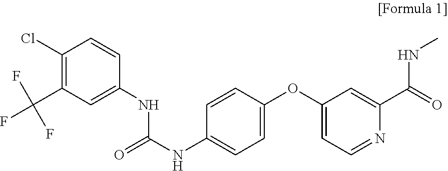 [SMILES: CNC(=O)c1cc(Oc2ccc(NC(=O)Nc3ccc(Cl)c(C(F)(F)F)c3)cc2)ccn1]

Generally, sorafenib represented by Formula 1 is known as an oral multikinase inhibitor that simultaneously inhibits receptor tyrosine kinases, which are expected to be overexpressed in tumor cells or tumor vessels, for example, VEGFR-2, PDGFR-β, and c-kit, and serine/threonine kinases in a signaling pathway, for example, Raf kinase.

Clinical trials for sorafenib efficacy on various solid tumors are in progress, and sorafenib is already used as a target anticancer agent for renal cell carcinoma. According to the progress of clinical trials on advanced hepatocellular carcinoma, recently, sorafenib has been approved by the US FDA as a therapeutic agent for hepatocellular carcinoma, which is impossible to be removed by excision.

However, as described above, there was a problem in that many patients are still identified as unresponsive with respect to sorafenib administration and treatment, and thus the treatment response may not be predicted and confirmed before initiation of the treatment. According to the method of predicting the response to sorafenib treatment using the SLC15A2 genetic polymorphism of the present invention, compared to the conventional sorafenib treatment for a liver cancer patient, it is possible to predict the response of a subject with respect to the sorafenib treatment, thereby administering a proper drug, and implementing selective treatment with an anticancer agent that can minimize side effects in liver cancer treatment. Further, liver cancer is only an example, and it should be obvious to those of ordinary skill in the art that the method according to the present invention can also be applied to diseases to which the sorafenib treatment may be applied.

Specifically, as shown in Table 2 of Example 1, a number of candidate genes associated with the sorafenib response in a liver cancer patient and coding variants thereof were identified. It was confirmed that, among 708 single-nucleotide variations (SNVs), 36 variants are located in genomic regions, and 15 variants are located in coding regions of nine genes. Such a result revealed the presence of polymorphisms in the sorafenib response-related genes.

From the 15 SNVs, it can be seen that 13 variations are located in the drug response-related genes, and two variations are sorafenib-target candidate genes. Drug response-related genes are genes associated with absorption, distribution, metabolism and excretion (ADME) of drugs. For more precise evaluation of sorafenib efficacy and equivalence, in selection of a sorafenib response-related gene from these genes, genetic information associated with the ADME of a drug was validated.

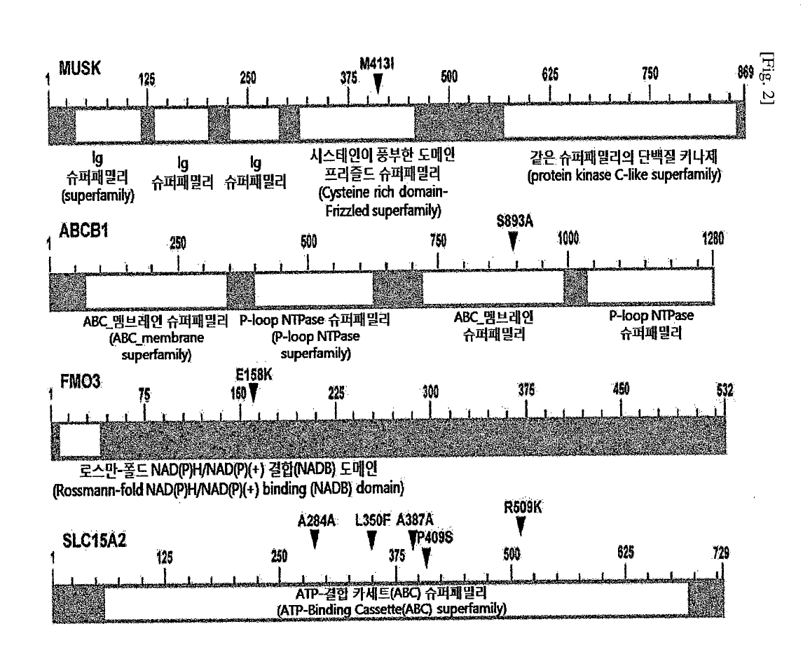

As shown in FIG. 2, it was seen that six encoded SNVs are non-synonymous variations that have the probability of damaging a protein-encoding function, and located in four genes, for example, a sorafenib-target candidate gene MUSK and ADME-related genes ABCB1, FMO3 and SLC15A2.

The polymorphism in the SLC15A2 gene of the present invention may be a C-to-T variation at the 501^{st} nucleotide in the SLC15A2 gene (NCBI ACESSION NO: NM_021082; SEQ. ID. NO: 4).

Overall clinical results according to the conventional cancer chemotherapy in which a proper anticancer agent is selected and administered according to the type and severity of cancer, not according to an individual cancer patient, have significant differences in the therapeutic effect of such cancer chemotherapy according to a patient, and to overcome the differences, various methods were suggested.

To overcome shortcomings of the chemotherapy, there was an attempt to select and administer a proper anticancer agent by analyzing SNPs per individual, and the response of a patient with respect to sorafenib treatment was able to be predicted by identifying the presence of polymorphisms in the SLC15A2 gene associated with the response to the sorafenib treatment for liver cancer patient of the present invention. Therefore, the polymorphism has a probability as a reliable biomarker that can predict the treatment response before sorafenib treatment is performed on liver cancer patients.

Specifically, as seen from Example 3, the usefulness of genetic variations in the SLC15A2 gene was confirmed.

Five coding variants were identified in the SLC15A2 gene by NGS analysis, and three non-synonymous SNVs, L350F, P409S and R509K, which may cause a functional alternation in gene product, were selected so as to analyze genotypes for 233 liver cancer patients that had received sorafenib treatment over 6 weeks.

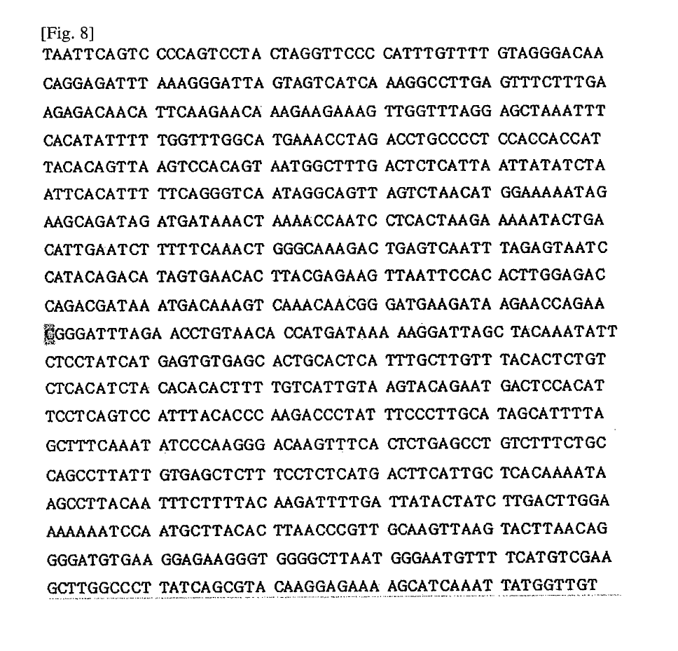

As a result, three SNP genotypes (C/C, C/T and T/T) were identified, and as shown in FIG. 2, when the nucleotide C (shown with fluorescence in FIG. 8) was substituted with T at position 501 of the nucleotide sequence of the SLC15A2 gene (NCBI ACESSION NO: NM_021082, SEQ. ID. NO: 4) associated with the response to sorafenib treatment, the presence of a C/T or T/T genotype showed longer progression-free survival time due to a higher response to sorafenib treatment than that of a subject with a C/C genotype.

The subject of the present invention may be a patient having the SLC15A2 gene, suffering from any disease, besides liver cancer, and preferably a liver cancer patient, and the sample may be at least one selected from the group consisting of a tissue sample, biopsy, blood, saliva, feces, cerebrospinal fluid, semen, tears and urine, which have the SLC15A2 gene, and preferably blood.

More specifically, the present invention provides a method of predicting the response to sorafenib treatment, which includes: obtaining a biological sample from a subject; determining if the 501^{st} nucleotide in the SLC15A2 gene (NCBI ACESSION NO: NM_021082; SEQ. ID. NO: 4) of the subject has a C/T or T/T genotype; and predicting the response of the subject with respect to sorafenib treatment based on the determination in order to resolve the above-described problem. Here, the presence of a C/T or T/T genotype is evaluated as superior in the response to the sorafenib treatment, compared to a subject with a C/C genotype.

In other words, to detect genetic variation, genomic DNA was isolated from the sample obtained from the subject, amplified by PCR, and analyzed by detecting if a C/T or T/T genotype is present at the 501^{st} nucleotide (shown in fluorescent green of FIG. 8) in the SLC15A2 gene (NCBI ACESSION NO: NM_021082, SEQ. ID. NO: 4) of the subject through an individual SNP assay, thereby detecting the absence or presence of the SLC15A2 genetic polymorphism.

First, the method includes obtaining a biological sample from a subject.

The term “biological sample” used herein refers to a sample from a patient, and includes a sample, for example, tissue, cells, whole blood, serum, blood plasma, saliva, sputum, cerebrospinal fluid or urine, which has a different expression level of a liver cancer marker gene, SLC15A2 gene, but the present invention is not limited thereto. Preferably, the sample is blood.

In this step, the sample may be extracted from a subject, and therefrom genomic DNA is obtained. A method of isolating the genomic DNA is not particularly limited, and may be a method known in the art. Commercially available DNA isolation kits may include, but are not limited to, for example, the Puregene DNA isolation kit (Gentra Systems, Inc.), the blood DNA isolation kit (2-032-805, Roche Diagnostics Corp.), the GenomicPrep blood DNA isolation kit (27-5236-01, Amersham Biosciences Corp.), the PAXgene blood DNA kit (761133, Qiagen Inc.), the GNOME whole blood DNA isolation kit (2011-600, Qbiogene Inc.) and the Wizard genomic DNA purification kit (A1120, Promega U.S.).

A region containing the SLC15A2 gene in the isolated genomic DNA may be amplified by PCR using primers (SEQ. ID. NO: 1 and SEQ. ID. NO: 2) shown below.

Also, besides the PCR amplification or Southern blotting, other nucleic acid amplification methods such as the ligase chain reaction (refer to the article [Abravaya, K. et al., Nucleic Acids Research, 23, 675-682, 1995]), branched DNA signal amplification (refer to the article [Jrdea, MS et al., AIDS, 7(supp. 2), S11-514, 1993]), isothermal nucleic acid sequence-based amplification (NASBA)(refer to the article [Kievits, T. et al., J. Virological., Methods 35, 273-286, 1991], and other self-sustained sequence replication assays may also be used.

Subsequently, the method of the present invention includes determining if the 501^{st} nucleotide in the SLC15A2 gene (NCBI ACCESSION NO: NM_021082; SEQ. ID. NO: 4) of the subject has a C/T or T/T genotype.

The determining of the genotype may be performed by a nucleic acid-based detection assay.

According to an exemplary embodiment of the present invention, an SLC15A2 genetic polymorphism sequence may be detected using direct sequencing. In such an analysis method, first, DNA samples are isolated from a subject using a proper method, and a region of interest is amplified by being cloned in a vector and then grown in host cells (e.g., bacteria). Following amplification, DNA in the region of interest (e.g., including SNPs or mutations of interest) is analyzed by a proper method, for example, manual sequencing using a radiation marker nucleotide or automatic sequencing, but the present invention is not limited thereto. The sequencing result is visualized using a proper method. By analyzing the sequence, the presence of predetermined SNPs or mutations are identified.

Also, according to an exemplary embodiment of the present invention, a variant sequence is detected by a PCR-based assay. In one embodiment, the PCR assay uses an oligonucleotide primer that is only hybridized with a variant or wild allele (e.g., in a polymorphism or mutation region). A DNA sample was amplified using a set of primers and analyzed.

Preferably, in the present invention, to determine a genotype, a set of primers set forth in SEQ. ID. NO: 1 and SEQ. ID. NO: 2 are used to amplify the SLC15A2 gene, and a nucleotide polymorphism present at the 501^{st} nucleotide in the SLC15A2 gene was detected by sequencing.

The prediction of the response of a subject with respect to sorafenib treatment according to the present invention may be performed by a method of analyzing the expression of DNA in the above-described step, for example, clustering algorithms or the SPSS statistical program, but the present invention is not limited thereto.

The clustering algorithms are analyzing methods for identifying basic gene sets, and may be effectively performed on a large group of profiles for which it is difficult to categorize expected characteristics. Methods of performing the clustering algorithms are known in the art and articles, for example, Fukunaga, 1990, Statistical Pattern Recognition, 2nd Ed., Academic Press, San Diego; Everitt, 1974, Cluster Analysis, London: Heinemann Educ. Books; Hartigan, 1975, Clustering Algorithms, New York: Wiley; Sneath and Sokal, 1973, Numerical Taxonomy, Freeman; Anderberg, 1973, Cluster Analysis for Applications, Academic Press: New York may be referenced.

Moreover, the detection of the SLC15A2 genetic polymorphism may be performed using a fluorescence based sequence detection system such as the ABI PRISM® 7900HT Sequence Detection System (AME Bioscience).

By comparing gene expression in liver cancer patients by the prediction method, treatment responses and effects with respect to the sorafenib treatment in the liver cancer patients may be predicted. In other words, according to the identification of the presence of the SLC15A2 genetic polymorphism, which is a marker of the present invention, from the liver cancer patient, it can be predicted that, when a C/T genotype in which C is changed into T at position 501 in the SLC15A2 gene or a T/T genotype is found, compared to a liver cancer patient with a C/C genotype, the liver cancer patient with the C/T or T/T genotype is more affected by and has a better response to the sorafenib treatment.

As a result, compared to conventional anticancer treatment, individually tailored treatment may be implemented by predicting the response to treatment with an anticancer agent, and thus the method according to the present invention may be an effective treatment method that can reduce side effects, costs and time for cancer treatment.

According to another exemplary embodiment of the present invention, a marker composition for predicting the response to sorafenib treatment, which includes an agent for detecting the absence or presence of the SLC15A2 genetic polymorphism affecting the response to sorafenib treatment and a diagnosis kit for predicting the response to sorafenib treatment, which includes the composition, are provided so as to resolve the above-described problem.

The agent for detecting the absence or presence of the SLC15A2 genetic polymorphism may include a set of primers set forth in SEQ. ID. NO: 1 and SEQ. ID. NO: 2, wherein the SLC15A2 genetic polymorphism is a C-to-T variation at the 501^{st} nucleotide in the SLC15A2 gene (NCBI ACESSION NO: NM_021082; SEQ. ID. NO: 4). When the 501^{st} nucleotide in the SLC15A2 gene (NCBI ACESSION NO: NM_021082; SEQ. ID. NO: 4) has a C/T or T/T genotype, compared to the case of a C/C genotype, it may be evaluated that the gene exhibits a stronger response to sorafenib.

A sample from a subject may be a minimum amount of blood obtained from a patient, specifically, the minimum amount of blood from which the minimum amount of DNA can be obtained so as to detect the absence or presence of the SLC15A2 genetic polymorphism, and more specifically, 3 to 6 ml of blood.

The diagnosis kit for predicting the response of a subject to the sorafenib treatment according to the present invention may be an RT-PCR kit or a DNA chip kit. The RT-PCR kit preferably includes a set of primers set forth in SEQ. ID. NO: 1 and SEQ. ID. NO: 2 that may specifically amplify mRNA of the SLC15A2 gene so as to include the 501^{st} nucleotide of the SLC15A2 gene (NCBI ACESSION NO: NM_021082; SEQ. ID. NO: 4), which is a liver cancer diagnostic marker.

The kit for detecting a marker according to the present invention may include a composition solution or device including primers for measuring an expression level of a liver cancer diagnostic marker, probes or an antibody selectively recognizing the marker, and one or more different components, which are suitable for an analysis method.

The RT-PCR kit may include a test tube or different suitable container, reaction buffers (pH and magnesium concentration are varied), deoxynucleotides (dNTPs), enzymes such as Taq-polymerase and reverse transcriptase, DNase, an RNase inhibitor, DEPC-water and sterilized water, in addition to a set of marker gene-specific primers designed by those of ordinary skill in the art. Also, as a quantification control, 18s rRNA was used, and therefore the RT-PCR kit may include a set of primers specific to the 18s rRNA.

Also, the kit of the present invention may be a kit for detecting a diagnostic marker, which includes essential factors that are required to run the DNA chip. The DNA chip kit may include a substrate to which cDNA corresponding to a gene or a fragment thereof is attached as a probe, and the substrate may include cDNA corresponding to a quantification control gene or a fragment thereof.

Preferably, the DNA chip kit includes primers or probes immobilized onto a substrate to specify a polymorphism at the 501^{st} nucleotide of the SLC15A2 gene (NCBI ACESSION NO: NM_021082; SEQ. ID. NO: 4), and a labeling means for detecting the hybridization between the DNA chip and a sample. Also, probes including a positive control hybridized with all nucleotide sequences in the sample and a negative control not hybridized with any nucleotide sequence may be bound to a surface of the substrate. These are used to examine if the hybridization efficiently takes place in the DNA chip, and a positive control and/or a negative control may be further included on the substrate.

The labeling means may be a fluorescent substance containing a biotin-binding protein, and an example of such a fluorescent substance may be streptavidin-R-phycoerythrin (s) or streptavidin-cyanine 3, but the present invention is not limited thereto.

Also, the diagnosis kit may further include an amplification means that can amplify DNA of the sample, and a means for selectively extracting a gene from a subject. A method of amplifying the sample DNA using PCR and a method of extracting the gene from the subject are known in the art, and thus detailed descriptions thereof will be omitted in the specification.

According to an exemplary embodiment of the present invention, the absence or presence of the nucleotide polymorphism of the SLC15A2 gene may be detected using hybridization analysis. In the hybridization analysis, the absence or presence of predetermined SNP or mutation is determined based on an ability of DNA in the sample, which can be hybridized with a complementary DNA molecule (e.g., an oligonucleotide probe). Various hybridization analyses using a variety of techniques for hybridization and detection thereof may be used.

First, by a direct detection method for hybridization, hybridization between a target sequence (e.g., SNP or mutation) and a probe may be directly detected by visualizing the binding probe (e.g., Northern or Southern blotting; refer to [Ausable et al. (eds.), Current Protocols in Molecular Biology, John Wiley & Sons, NY(1991)]). In such analyses, genomic DNA (Southern) or RNA (Northern) is isolated from a subject. Then, the DNA or RNA is cleaved with a series of restriction enzymes that randomly cleave a genome, and then an arbitrary marker is analyzed. Afterward, DNA or RNA is isolated (e.g., on an agarose gel) and transferred to a membrane. A probe or probes specifically labeled (e.g., introduction of a radiation-labeled nucleotide, etc.) with respect to SNPs or mutations to be detected may contact to the membrane under a condition, or a lowly, moderately or highly stringent condition. Non-binding probes are removed, and binding is detected by visualizing the labeled probes.

Also, in the present invention, a hybridization detecting method using “DNA chip” analysis may be used. A variant sequence is detected using the DNA chip hybridization analysis. In this analysis, a series of oligonucleotide probes are immobilized to a solid-phase scaffold. The oligonucleotide probes are manufactured to be specific to predetermined SNPs or mutations. The DNA sample of interest is in contact with the “DNA chip” and then a resulting hybrid is detected.

The DNA chip technique uses a high density microarray of oligonucleotide probes, which are immobilized to the “chip.” A probe analysis is manufactured through a photo-direct chemical analysis process (Affymetrix), which is produced by combining a photolithography process technique used in the semiconductor industry and dry chemistry analysis. A chip-exposed region is limited using a series of photolithographic masks, followed by specific chemical analysis. A high density oligonucleotide array containing respective probes located at previous determined positions is manufactured by such a process. A plurality of probe arrays are simultaneously synthesized on a great quantity of glass wafers. Subsequently, the wafer is diced, each probe array is packaged with an injection molding plastic cartridge to protect the probes from the surroundings, and provided to a chamber for hybridization.

A nucleic acid to be analyzed is isolated, amplified by PCR, and labeled with a fluorescent reporter group. Subsequently, the labeled DNA is subjected to a reaction with the array at a constant temperature using Fluidics Station. Subsequently, the array is inserted into a scanner, so as to detect a hybridization pattern. A hybridization result is obtained by collection using light emitted from the fluorescent reporter group introduced in advance to a target, the fluorescent reporter group binding to the probe array. The probe perfectly matching the target generally emits a stronger signal than a mismatched probe. Since the position and sequence of each probe on the array are already known, the target nucleic acid applied to the probe array can be identified through complementarity.

The term “primer” used herein refers to a strand of short nucleic acid sequences having a free 3′-end hydroxyl group, which can form base pairs with a complementary template and serves as a starting point for replicating a template strand. The primer may start DNA synthesis in the presence of reagents for polymerization (that is, DNA polymerase or reverse transcriptase) and four different nucleoside triphosphates in proper buffer solutions at a proper temperature. In the present invention, PCR amplification may be carried out using sense and antisense primers of a UQCRH polynucleotide so as to diagnose liver cancer based on the production of a desired product. PCR conditions, and the lengths of sense and antisense primers may be modified based on what is known in the art.

The term “probe” used herein refers to a fragment of a nucleic acid such as RNA or DNA corresponding to several to hundreds of bases that can achieve specific binding to mRNA, and may be labeled to identify the presence of specific mRNA.

Probes may be manufactured in forms of an oligonucleotide probe, a single-stranded DNA probe, a double-stranded DNA probe, an RNA probe, etc. In the present invention, hybridization may be performed using a probe complementary to the UQCRH polynucleotide, and liver cancer may be diagnosed from a hybridization result. Selection of proper probes and hybridization conditions may be modified based on what is known in the art.

The primer or probe of the present invention may be chemically synthesized using a phosphoramidite solid scaffold method or other well-known methods. Such nucleic acid sequences may also be modified by various means known in the art. Non-limiting examples of such modifications include methylation, capping, substitution of one or more analogues of natural nucleotides, and nucleotide variation, for example, variation to non-charged linkages (for example: methyl phosphonate, phosphotriester, phosphoroamidate, carbamates, etc.) or charged linkages (for example: phosphorothioate, phosphorodithioate, etc.).

Hereinafter, the present invention will be described in further detail with respect to examples. These examples are merely provided to exemplify the present invention, and thus it should be construed that the scope of the present invention is not limited by the examples.

### EXAMPLES

### Example 1

### Identification of Sorafenib Response-related Genes

To predict the response to sorafenib, various single-nucleotide variations (SNVs) and genes, which were associated with the sorafenib responses, were identified.

To identify the sorafenib response-related SNVs, genomic patterns were identified through next-generation sequencing (NGS) performed on genomes of seven patients receiving sorafenib treatment (four: strong responders, three: poor responders).

Specifically, genomic DNA of a patient was extracted from leukocytes of a patient using a MagAttract DNA blood Midi Kit (Qiagen, Inc. Valencia, Calif., USA) according to a user manual of the kit. Also, DNA quality was assessed using a Nanodrop spectrometer (Nanodrop Technologies, Wilmington, DE, USA), and 5 μg of the genomic DNA was sheared using a Covaris S series ultrasonicator (Covaris, Woburn, Mass., USA). Fragments of the sheared genomic DNA were end-repaired, A-tailed and ligated to pair-end adapters (Pair End Library Preparation Kit, Illumina, Calif., USA), and then amplified according to a user manual for PCR. The quality of a library and a DNA concentration were measured using an Agilent 2100 BioAnalyzer (Agilent, Santa Clara, Calif., USA), and quantified using an SYBR green qPCR protocol for LightCycler 480 (Roche, Indianapolis, Ind., USA) according to Illumina's library quantification protocol. Paired-end sequencing (2×100 bp) was performed on Illumina HiSeq 2000 using HiSeq Sequencing kits.

A 90-bp paired-end sequence was read together with 300-bp inserted into a hp19 human reference genome (NCBI build 37) using BWA algorithm1 ver. 0.5.9. Also, two mismatches were allowed in a 45-bp seed sequence, and a SAM tool was used to remove PCR duplicates of the sequence reads, which had been performed during the library formation process. The reads adjusted by the tool realigned positions estimated as insertions/deletions (indel) with an improved mapping quality using the GATK Indel Realigner algorithm (Kanehisa M (2002) The KEGG datanucleotide. Novartis Foundation Symposium 247: 91-101; discussion 101-103, 119-128, 244-152.).

Also, SNP genotyping performed to confirm the NGS analysis was performed with an Axiom genotyping solution using an Axiom Genome-Wide ASI 1 Array Plate (Affymetrix, Santa Clara, Calif., USA). Here, a reagent kit used herein was used according to a user manual. Further, total genomic DNA (200 ng) was used, and the genotyping result was utilized using Genotyping Console 4.1 (Affymetrix) and Axiom GT1 algorithms according to a user manual of the algorithms.

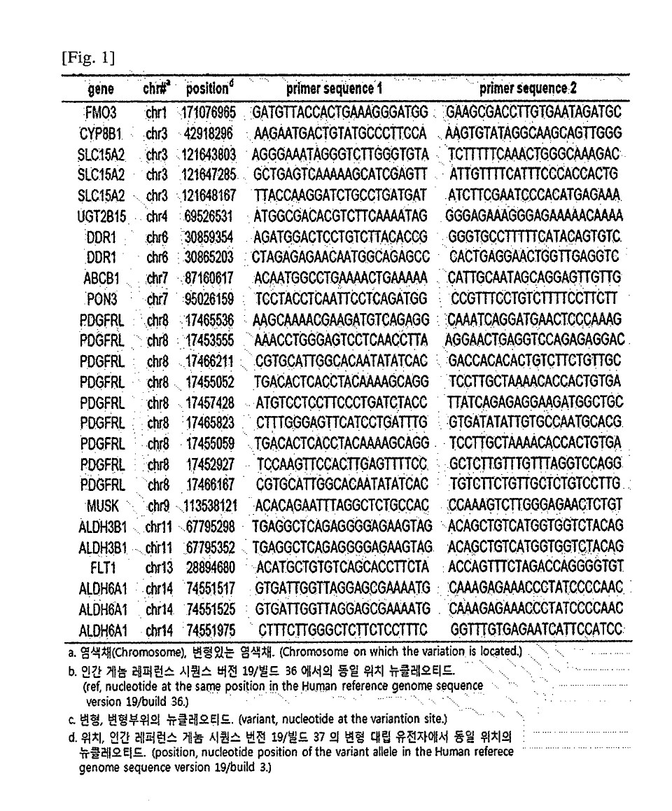

In addition, the sequence was analyzed using an automatic sequencer ABI 3730 (Applied Biosystems, Carlsbad, Calif., USA), and a target region was amplified by PCR. Details of the PCR and primer sequences are shown in Table 1 and FIG. 1.

The PCR was carried out in a thermal cycler (PTC-100; MJ Research. Inc, USA) under the following conditions: 3-minute predenaturation at 94° C., 30 cycles of 1-minute denaturation at 94° C., 1-minute annealing at 55° C. and 4-minute extension at 72° C., and 10-minute additional reaction at 72° C. Subsequently, to remove polymerases and non-specific amplified products, following centrifugation in an agarose gel, a desired band of amplified product was fragmented and purified using a gel extraction kit (Geneall, Korea).

In addition, the sorafenib response-related genes identified by the above-described method are listed in Table 2.

TABLE 1

| gene | chr# | position | primer sequence 1 | | primer sequence 2 | |
|---|---|---|---|---|---|---|
| FMO3 | chr1 | 171076965 | GATGTTACCACTGAAAGGGATGG | SEQ ID NO. 5 | GAAGCGACCTTGTGAATAGATGC | SEQ ID NO. 6 |
| CYP8B1 | chr3 | 42918296 | AAGAATGACTGTATGCCCTTCCA | SEQ ID NO. 7 | AAGTGTATAGGCAAGCAGTTGGG | SEQ ID NO. 8 |
| SLC15A2 | chr3 | 121643803 | AGGGAAATAGGGTCTTGGGTGTA | SEQ ID NO. 9 | TCTTTTTCAAACTGGGCAAAGAC | SEQ ID NO. 10 |
| SLC15A2 | chr3 | 121647285 | GCTGAGTCAAAAAGCATCGAGTT | SEQ ID NO. 11 | ATTGTTTTCATTTCCCACCACTG | SEQ ID NO. 12 |
| SLC15A2 | chr3 | 121648167 | TTACCAAGGATCTGCCTGATGAT | SEQ ID NO. 13 | ATCTTCGAATCCCACATGAGAAA | SEQ ID NO. 14 |
| UGT2B15 | rhr4 | 69596531 | ATGGCGACACGTCTTCAAAATAG | SEO ID NO. 15 | GGGAGAAAGGGAGAAAAACAAAA | SEQ ID NO. 16 |
| DDR1 | chr6 | 30859354 | AGATGGACTCCTGTCTTACACCG | SEQ ID NO. 17 | GGGTGCCTTTTTCATACAGTGTC | SEQ ID NO. 18 |
| DDR1 | chr6 | 30865203 | CTAGAGAGAACAATGGCAGAGCC | SEQ ID NO. 19 | CACTGAGGAACTGGTTTGAGGTC | SEQ ID NO. 20 |
| ABCB1 | chr7 | 87160617 | ACAATGGCCTGAAAACTGAAAAA | SEQ ID NO. 21 | CATTGCAATAGCAGGAGTTGTTG | SEQ ID NO. 22 |
| PON3 | chr7 | 95026159 | TCCTACCTCAATTCCTCAGATGG | SEQ ID NO. 23 | CCGTTTCCTGTCTTTTCCTTCTT | SEQ ID NO. 24 |
| PDGFRL | chr8 | 17465536 | AAGCAAAACGAAGATGTCAGAGG | SEQ ID NO. 25 | CAAATCAGGATGAACTCCCAAAG | SEQ ID NO. 26 |
| PDGFRL | chr8 | 17453555 | AAACCTGGGAGTCCTCAACCTTA | SEQ ID NO. 27 | AGGAACTGAGGTCCAGAGAGGAC | SEQ ID NO. 28 |
| PDGFRL | chr8 | 17466211 | CGTGCATTGGCACAATATATCAC | SEQ ID NO. 29 | GACCACACACTGTCTTCTGTTGC | SEQ ID NO. 30 |
| PDGFRL | chr8 | 17455052 | TGACACTCACCTACAAAAGCAGG | SEQ ID NO. 31 | TCCTTGCTAAAACACCACTGTGA | SEQ ID NO. 32 |
| PDGFRL | chr8 | 17457428 | ATGTCCTCCTTCCCTGATCTACC | SEQ ID NO. 33 | TTATCAGAGAGGAAGATGGCTGC | SEQ ID NO. 34 |
| PDGFRL | chr8 | 17465823 | CTTTGGGAGTTCATCCTGATTTG | SEQ ID NO. 35 | GTGATATATTGTGCCAATGCACG | SEQ ID NO. 36 |
| PDGFRL | chr8 | 17455059 | TGACACTCACCTACAAAAGCAGG | SEQ ID NO. 37 | TCCTTGCTAAAACACCACTGTGA | SEQ ID NO. 38 |
| PDGFRL | chr8 | 17452927 | TCCAAGTTCCACTTGAGTTTTCC | SEQ ID NO. 39 | GCTCTTGTTTGTTTAGGTCCAGG | SEQ ID NO. 40 |
| PDGFRL | chr8 | 17466167 | CGTGCATTGGCACAATATATCAC | SEQ ID NO. 41 | TGTCTTCTGTTGCTCTGTCCTTG | SEQ ID NO. 42 |
| MUSK | chr9 | 113538121 | ACACAGAATTTAGGCTCTGCCAC | SEQ ID NO. 43 | CCAAAGTCTTGGGAGAACTCTGT | SEQ ID NO. 44 |
| ALDH3B1 | chr11 | 67795298 | TGAGGCTCAGAGGGGAGAAGTAG | SEQ ID NO. 45 | ACAGCTGTCATGGTGGTCTACAG | SEQ ID NO. 46 |
| ALDH3B1 | chr11 | 67795352 | TGAGGCTCAGAGGGGAGAAGTAG | SEQ ID NO. 47 | ACAOCTGTCATGGTGGTCTACAG | SEQ ID NO. 48 |
| FLTI | chr13 | 28894680 | ACATGCTGTGTCAGCACCTTCTA | SEQ ID NO. 49 | ACCAGTTTCTAGACCAGGGGTGT | SEQ ID NO. 50 |
| ALDH6A1 | chr14 | 74551517 | GTGATTGGTTAGGAGCGAAAATG | SEQ ID NO. 51 | CAAAGAGAAACCCTATCCCCAAC | SEQ ID NO. 52 |
| ALDH6A1 | chr14 | 74551525 | GTGATTGGTTAGGAGCGAAAATG | SEQ ID NO. 53 | CAAAGAGAAACCCTATCCCCAAC | SEQ ID NO. 54 |
| ALDH6A1 | chr14 | 74551975 | CTTTCTTGGGCTCTTCTCCTTTC | SEQ ID NO. 55 | GGTTTGTGAGAATCATTCCATCC | SEQ ID NO. 56 |

In Table 1, chr is the abbreviation of a chromosome, and chr# represents a variation position of a chromosome. Also, the position shown in Table 1 refers to a nucleotide position in a variant allele of the human reference genome sequence version 19/build 37.

TABLE 2

| | | | | | Ref | Variant |
|---|---|---|---|---|---|---|
| | | | | | amino | amino |
| Gene | Chromosome^{a} | Position^{b} | Ref^{c} | Variant^{d} | acid^{e} | acid^{f} |
| ABCB1 | ch7 | 87160618 | A | C | S | A |
| ALDH3B1 | chr11 | 67795299 | G | A | P | P |
| ALDH3B1 | chr11 | 67795353 | G | A | L | L |
| CYP21A2 | chr6 | 32006317 | C | T | L | L |
| DDR1 | chr6 | 30865204 | A | C | P | P |
| FMO3 | chr1 | 171076966 | G | A | E | K |
| MUSK | chr9 | 113538122 | G | A | M | I |
| SLC15A2 | chr3 | 121646641 | A | G | A | A |
| SLC15A2 | chr3 | 121643804 | C | T | L | F |
| SLC15A2 | chr3 | 121641693 | G | A | A | A |
| SLC15A2 | chr3 | 121647286 | C | T | P | S |
| SLC15A2 | chr3 | 121648168 | G | A | R | K |
| SLC22A15 | chr1 | 116534852 | C | T | S | S |
| SLC7A7 | chr14 | 23282449 | C | T | S | S |
| SLC7A7 | chr14 | 23382110 | A | G | I | I |

^{a}Chromosome on which the variation is located.
^{b}Nucleotide position of the variant allele in the human reference genome sequence version 19/build 36.
^{c}Nucleotide at the same position in the human referece genome sequence version 19/build 36.
^{d}Nucleotide at the variantion site.
^{e}Amino acid encoded by the corresponding codon in the reference sequence.
^{f}Amino acid encoded by the corresponding codon in the variant sequence.
^{g}Genotypes of good responders.
^{h}Genotypes of poor responders.

As confirmed from Table 2, a number of candidate genes associated with the sorafenib response and coding variants thereof were identified in liver cancer patients. Specifically, it was identified that, among 708 SNVs, 36 variations were located in genomic regions, and 15 SNVs were located in coding regions of 9 genes. Accordingly, the presence of polymorphisms of sorafenib response-related genes was confirmed.

### Example 2

### Confirmation of Polymorphism of SLC15A2 Gene

The presence of polymorphisms of sorafenib response-related genes was confirmed according to Example 1, and it can be seen that 13 of the 15 SNVs shown in Table 1 are located in a drug response-related gene, but 2 variations are sorafenib target candidate genes.

The drug response-related gene is a gene associated with the ADME of a drug, and for more precise evaluation of sorafenib efficacy and equivalence, genetic information associated with the ADME of a drug was identified to sort sorafenib response-related genes.

Specifically, each SNV in which a polymorphism is present in a sorafenib response-related gene, identified in Example 1, was found on an UCSC gene table according to genomic characteristics such as a coding region, an untranslated region (UTR) and an unexpressed region (intron). Non-synonymous SNV information was extracted by comparing UCSC (http://genome.ucsc.edu/) reference gene information. Results are shown in FIG. 2.

As shown in FIG. 2, 6 encoded SNVs were identified as non-synonymous variations, which may damage a protein encoding function, and all of them were located in four genes including a sorafenib-target candidate gene MUSK and ADME-related genes ABCB1, FMO3 and SLC15A2.

### Example 3

### Prediction of Response to Sorafenib Treatment using Polymorphism of SLC15A2 Gene

SLC15A2 is a member of the membrane transport protein group, involved in drug delivery. Genetic variation efficiency of the SLC15A2 gene was investigated.

Five coding variants were identified in the SLC15A2 gene by NGS analysis, and three non-synonymous SNVs (L350F, P409S and R509K), which may cause a functional alternation in gene product, were selected so as to analyze genotypes for 233 liver cancer patients that had received sorafenib treatment over 6 weeks.

Specifically, for structural analyses of variations in SLC15A2, NCBI ACESSION NO: NM_001145998 was used. Also, a three-dimensional structure was constructed using Phyre 2.0 (http://www.sbg.bio.ic.ac.uk/phyre2/html/page.cgi?id=index), and post-translational modification was analyzed using KinasePhos (kinasephos.mbc.nctu.edu.tw). Further, pathway analysis was performed for genes annotated to harbor significant mutations using Kyoto Encyclopedia of Genes and Genomes (KEGG; 32, http://www.genome.jp/kegg/) and Biocarta (http://www.biocarta.com/). Moreover, together with publicly available data and pathway analyses for SLC15A2, described above, integrative analysis was performed by the cBioPortal website (www.cbioportal. org).

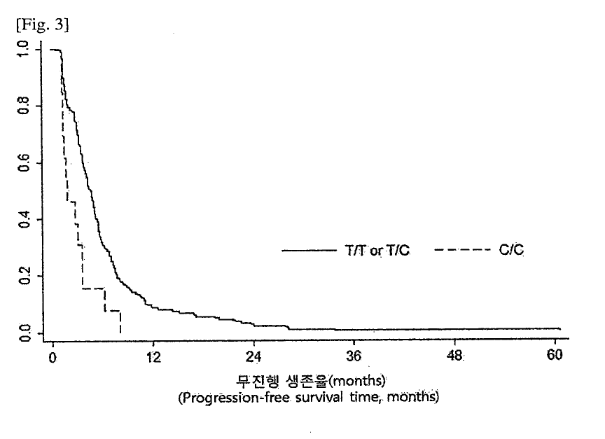

Also, to confirm SNPs to be analyzed, 249 patients treated with sorafenib were genotyped using the MassARRAY system (Sequenom, San Diego, Calif., USA), thereby obtaining three SNP genotypes (C/C, C/T and T/T). Progression-free survival (PFS) was evaluated by the Kaplan-Meier method, and the result is shown in FIG. 3.

Specifically, the association between generic polymorphisms and risk for progression was assessed by a Cox proportional hazard model with adjustment by stage of hepatocellular carcinoma (HCC). Also, analyses of data obtained were performed using STATA version 10.1 (Stata Corp, College Station, Tex., USA).

As shown in FIG. 3, in terms of the response to sorafenib treatment, when nucleotide C was substituted with T at position 501 in the SLC15A2 gene (NCBI ACESSION NO: NM_021082; SEQ. ID. NO: 4), the presence of a C/T or T/T genotype revealed that the subject exhibited a stronger response to sorafenib treatment than that with a C/C genotype, and thus had a longer increased progression-free survival (accumulative hazard ratio): 2.46; 95% confidence interval: 1.36˜4.44; P=0.003).

Therefore, it was confirmed that the SLC15A2 gene plays an important role in the response to sorafenib treatment for liver cancer patients, and thus is available as a reliable biomarker for predicting the response to sorafenib treatment.

### Example 4

### Functional Effect by Polymorphism in SLC15A2 Gene

To validate nucleotide polymorphisms in the SLC15A2 gene, functional analyses were performed on human liver cancer cell lines. As a result, in the Hep3B, SNU182 and PLC/PRFS cell lines, the present of nucleotide polymorphism at position 501 in the SLC15A2 gene (NCBI ACESSION NO: NM_021082; SEQ. ID. NO: 4) was validated, and sorafenib responses and SLC15A2 protein expression levels were measured for the respective cell lines.

First, human hepatocellular carcinoma (HCC)-derived cell lines such as Hep3B, SNU182 and PLC/PRFS cell lines were purchased from Korean Cell Line Bank (KCLB, Seoul, Republic of Korea), and genomic DNA was extracted from the cell lines derived from human HCC using a MagAttract DNA mini M48 kit (Qiagen) according to a user manual of the kit.

The extracted DNA was amplified by PCR using forward and reverse primers as described below. The PCR amplification was performed using a thermal cycler (PTC-100; MJ Research. Inc, USA) under the following conditions: 3-minute predenaturation at 94° C., 30 cycles of 1-minute denaturation at 94° C., 1-minute annealing at 55° C. and 4-minute extension at 72° C., and a 10-minute additional reaction at 72° C. Subsequently, to remove a polymerase and non-specific amplified products, following electrophoresis in an agarose gel, a desired band of amplified product was fragmented and purified using a gel extraction kit (Geneall, Korea).

Forward primer (SEQ. ID. NO: 1):
5′-GGGTCTTGGGTGTAAATGGA-3′
Reverse primer (SEQ. ID. NO: 2):
5′-CACACTTGGAGACCAGACGA-3′

A nucleotide sequence of each of the amplified products was analyzed by Sanger sequencing, and as shown in FIG. 4, the Hep3B, PLC/PRFS and SNU182 cell lines were identified as C/C, C/T and T/T genotypes, respectively.

Afterward, to confirm the sorafenib response in each cell line according to the SLC15A2 gene nucleotide polymorphism, each cell line was cultured in RPMI-1640 (Invitrogen, Carlsbad, Calif., USA) with 10%(v/v) fetal bovine serum (FBS) and 100 U/ml penicillin-streptomycin at 37° C. in 5% CO₂. Next, for the MTT assay, cells obtained by the above-described culture were plated on 96-well plates at a density of 1×10⁴ cells/well and treated with sorafenib for 48 hours in the RPMI-1640 used above.

Then, the number of viable cells was measured by performing the MTT assay (Promega Fitchburg, Wis., USA) according to a user manual of the MTT assay.

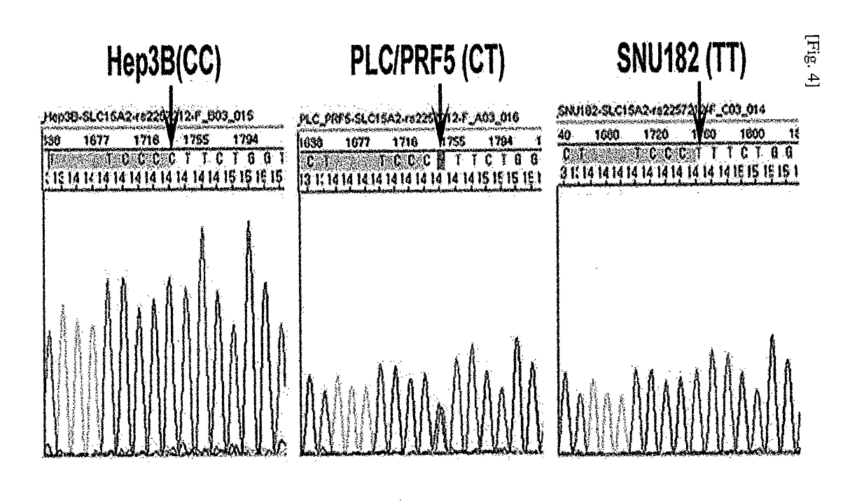

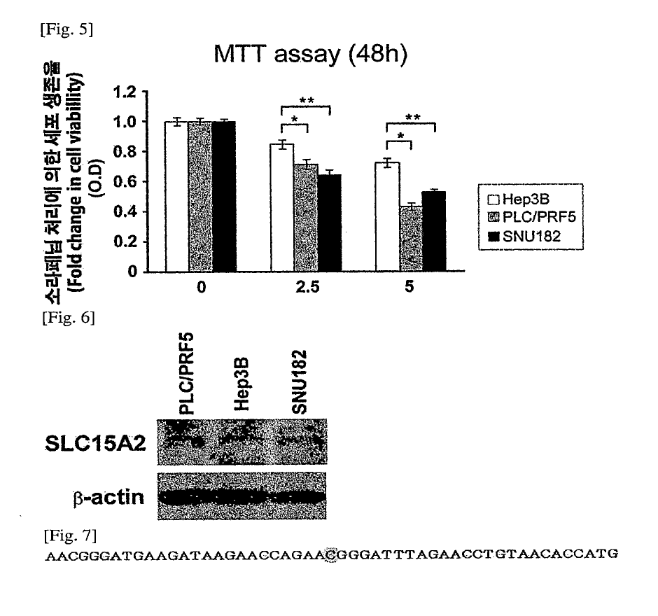

As shown in FIG. 5, although the proliferation of all of three types of cell lines was dose-dependently inhibited by sorafenib, it was confirmed that the SNU182 cell line with the T/T genotype and the PLC/PRFS cell line with the C/T genotype exhibited a stronger response to sorafenib than the Hep3B cell line with the C/C genotype.

Further, to investigate an influence of the SLC15A2 gene nucleotide polymorphism on SLC15A2 protein expression, SLC15A2 protein expression levels in the respective cell lines were confirmed by western blot analysis.

The western blot analysis was performed by a general method known in the art, 30 μg of cell lysates of each cell line culture above were loaded in 12% NuPage gel (Invitrogen) for SDS-PAGE, and transferred onto a membrane for western blotting, which is Immobilon (Millipore, Billerica, Mass., USA). Afterward, immunoblotting was performed using an anti-SLC15A2-primary antibody (Santa Cruz Biotechnology, Santa Cruz, Calif., USA) and anti-β-actin (Abcam, Cambridge, Mass., USA). Protein bands were detected using WestZol (iNtRon, Gyeonggi, Republic of Korea).

As shown in FIG. 6, different from the cell viability results, it was confirmed that similar protein expression levels of the SLC15A2 gene were observed regardless of SNP genotype.

That is, it is determined that the change in response to sorafenib according to the SLC15A2 gene nucleotide polymorphism is a functional change caused by a structural change, not by the change in expression level of SLC15A2 protein.

Therefore, according to a method of predicting the response to sorafenib treatment using genetic polymorphism of the present invention, a patient group with high responsiveness to sorafenib and thus exhibiting good prognosis may be selected, whereby responses to sorafenib treatment in liver cancer patients can be predicted. As a result, it can be expected that achievement of an optimal therapeutic effect by administering a proper drug to a liver cancer patient, reduction in the inconvenience of the patient, and reduction in treatment costs lead to an excellent anticancer effect. Also, selective treatment with an anticancer agent, which may minimize side effects of cancer treatment, and individually tailored chemotherapy can be implemented.

1. A method of predicting the response to sorafenib treatment, comprising:
obtaining a sample from a subject, and detecting the absence or presence of an SLC15A2 genetic polymorphism.

2. The method of claim 1, wherein the SLC15A2 genetic polymorphism is a C-to-T variation at the 501^{st} nucleotide in an SLC15A2 gene (NCBI ACESSION NO: NM_021082; SEQ. ID. NO: 4).

3. The method of claim 1, wherein the subject is a liver cancer patient, and the sample is blood.

4. The method of claim 1, wherein the method comprises:
obtaining a sample from a subject, and determining if the 501^{st} nucleotide in an SLC15A2 gene (NCBI ACESSION NO: NM_021082; SEQ. ID. NO: 4) of the subject has a C/T or T/T genotype; and predicting the response of the subject with respect to sorafenib treatment based on the determination, wherein the presence of the C/T or T/T genotype is evaluated as superior in response to sorafenib treatment, compared to a subject with a C/C genotype.

5. The method of claim 4, wherein the determining of a genotype comprises amplifying the SLC15A2 gene using a set of primers set forth in SEQ. ID. NO: 1 and SEQ. ID. NO: 2, and detecting single-nucleotide polymorphisms (SNPs) present in the 501^{st} nucleotide in the SLC15A2 gene by sequencing.

6. A marker composition for predicting the response to sorafenib treatment, comprising:
an agent for detecting the absence or presence of an SLC15A2 genetic polymorphism.

7. The marker composition of claim 6, wherein the SLC15A2 genetic polymorphism is a C-to-T variation at the 501^{st} nucleotide in an SLC15A2 gene (NCBI ACESSION NO: NM_021082; SEQ. ID. NO: 4).

8. The marker composition of claim 6, wherein the agent for detecting the absence or presence of the SLC15A2 genetic polymorphism comprises a set of primers set forth in SEQ. ID. NO: 1 and SEQ. ID. NO: 2.

9. The marker composition of claim 6, wherein, when the 501^{st} nucleotide in the SLC15A2 gene (NCBI ACESSION NO: NM_021082; SEQ. ID. NO: 4) has a C/T or T/T genotype, it is evaluated that a superior response to sorafenib treatment is exhibited, compared to a subject with a C/C genotype.

10. A diagnosis kit for predicting the response to sorafenib treatment, comprising:
the marker composition of claim 6.

11. The diagnosis kit of claim 10, which is an RT-PCR kit or a DNA chip kit.

12. The diagnosis kit of claim 11, wherein the DNA chip kit comprises primers or probes that are immobilized to a substrate, so as to detect a polymorphism at the 501^{st} nucleotide in an SLC15A2 gene (NCBI ACESSION NO: NM_021082; SEQ. ID. NO: 4), and may include a labeling means for detecting hybridization between the DNA chip and a sample.

13. The diagnosis kit of claim 12, wherein probes comprising a positive control hybridized with all nucleotide sequences in the sample and a negative control not hybridized with any nucleotide sequence are bound to a surface of the substrate.
